(19) United States
(12) Patent Application Publication
(10) Pub. No.: US 2015/0231015 A1
(43) Pub. Date: Aug. 20, 2015

(54) HUNGER CONTROL ABDOMINAL BINDER

(71) Applicants: Babs Adelaide Soranno, Akron, OH (US); Gina Dianne Smith, Akron, OH (US); Timothy Hayes, Lakewood, OH (US)

(72) Inventors: Babs Adelaide Soranno, Akron, OH (US); Gina Dianne Smith, Akron, OH (US); Timothy Hayes, Lakewood, OH (US)

(73) Assignee: INNOVATIVE SISTERS, LLC, Bath, OH (US)

(21) Appl. No.: 14/619,869

(22) Filed: Feb. 11, 2015

Related U.S. Application Data
(60) Provisional application No. 61/966,000, filed on Feb. 14, 2014.

(51) Int. Cl. A61H 1/00; A61F 5/00

(52) U.S. Cl. CPC A61H 1/008; A61F 5/0003

(57) ABSTRACT

A hunger control abdominal binder may include a garment adapted to be worn about a midsection of a human subject. Embodiments may further include a pressure applicator element having a generally flat base portion and a generally round convex portion extending from a plane of the base portion. The pressure applicator may be reversibly attachable to a plurality of positions on the garment, and the pressure applicator may be positionable between the midsection of the human subject and the garment so that the garment and pressure applicator cooperate to apply pressure to the midsection.

This application claims the benefit of U.S. provisional patent application No. 61/966,000 filed on Feb. 14, 2014 which is incorporated herein by reference in its entirety.

### I. BACKGROUND OF THE INVENTION

A. Field of Invention

The invention generally relates to the field of hunger control devices.

B. Description of the Related Art

It is known that hunger may be suppressed by applying pressure to particular regions of the abdomen. Doing so causes the brain to perceive that the stomach is full in a manner similar to the action of a gastric sleeve or certain gastric implants, but without the need for surgery. Other devices not requiring surgery are also known, including devices that operate by applying pressure to the abdomen. However, existing devices have a number of problems. For instance, existing devices tend to be very uncomfortable. Often the device irritates the user's skin by applying an uneven pressure which may result in digging into the user's flesh or causing abrasions.

Additionally, existing devices offer limited options for adjusting the device to a particular user's anatomy and body measurements. A single one-dimensional adjustment, such as by tightening or loosening a belt is not sufficient to optimally fit the device to all users, and thus the device's efficacy will be diminished. Moreover, existing devices are frequently difficult to hide under clothing because they tend to create relief patterns in overlaying clothing. Since the device is only effective while it's being worn, it is necessary to wear it during most if not all of the user's waking hours. But, if the device cannot be adequately hidden under clothing wearing the device in public without drawing attention would be difficult. Accordingly, efficacy may be further diminished by extended periods of non-use.

Some embodiments of the present invention may provide one or more benefits or advantages over the prior art.

### II. SUMMARY OF THE INVENTION

Embodiments may relate to a hunger control device, comprising: a garment adapted to be worn about a midsection of a human subject; and a pressure applicator element having a generally flat base portion and a generally round convex portion extending from a plane of the base portion, wherein the pressure applicator is reversibly attachable to a plurality of positions on the garment, and wherein the pressure applicator is positionable between the midsection of the human subject and the garment so that the garment and pressure applicator cooperate to apply pressure to the midsection.

Other embodiments may relate to a hunger control device, comprising: a pressure applicator element co-operable with an arbitrary corset, the pressure applicator having a generally flat base portion and a generally round convex portion extending from the plane of the base portion, wherein the pressure applicator is reversibly attachable to a plurality of positions on the garment, and wherein the pressure applicator is positionable between the midsection of the human subject and the garment so that the garment and pressure applicator cooperate to apply pressure to the midsection.

Embodiments may also relate to a pressure applicator, comprising: a generally flat base portion; a generally round convex portion extending from a plane of the base portion; means for reversibly attaching the pressure applicator to any of a plurality of positions on an arbitrary corset, wherein the pressure applicator is positionable between an abdomen of a human subject and the corset so that the corset and pressure applicator cooperate to apply pressure to the midsection; and a bag member adapted to receive the generally flat base portion and generally round convex portion the bag member having a hook strip of a hook-and-loop fastener structure disposed on a surface of the bag member for reversibly attaching it to the plurality of positions on the arbitrary corset; wherein the pressure applicator is adapted to minimize or eliminate relief patterns in overlaying garments resulting from its presence under the garment, said adaptation comprising the base portion of the pressure applicator having a curvature approximating the curvature of a wearer's midsection, the base portion having rounded corners, and the base portion lacking a brim structure.

Other benefits and advantages will become apparent to those skilled in the art to which it pertains upon reading and understanding of the following detailed specification.

### III. BRIEF DESCRIPTION OF THE DRAWINGS

The invention may take physical form in certain parts and arrangement of parts, embodiments of which will be described in detail in this specification and illustrated in the accompanying drawings which form a part hereof and wherein:

FIG. 1A is an elevation view of a first pressure applicator;

FIG. 1B is a bottom view of the first pressure applicator;

FIG. 1C is a front view of the first pressure applicator;

FIG. 2A is an elevation view of a second pressure applicator;

FIG. 2B is a bottom view of the second pressure applicator;

FIG. 2C is a front view of the second pressure applicator;

FIG. 3 is a bottom perspective view of a pressure applicator equipped with a hook fastener strip;

FIG. 4A is an elevation view of a pressure applicator partially inserted in a bag member;

FIG. 4B is a bottom view of a the pressure applicator of FIG. 4A; and

FIG. 5 is a perspective view of a pressure applicator installed in a garment member.

### IV. DETAILED DESCRIPTION OF THE INVENTION

Embodiments of the invention may include hunger control devices adapted to apply pressure to user-selected areas of a person's midsection. In general, embodiments include a garment adapted to be worn about a person's midsection, and a pressure applicator element which is positionable between the garment and the wearer. Devices as described herein may be used by affixing the pressure applicator to a selected position on the garment, aligning the pressure applicator with the wearer's abdomen, and fastening and/or tightening the garment to the wearer's midsection. Thus, the garment and the pressure applicator cooperate to apply pressure to a selected region of the wearer's abdomen thereby suppressing hunger. Suitable garments may include one or more of, without limitation, a corset or a belt.

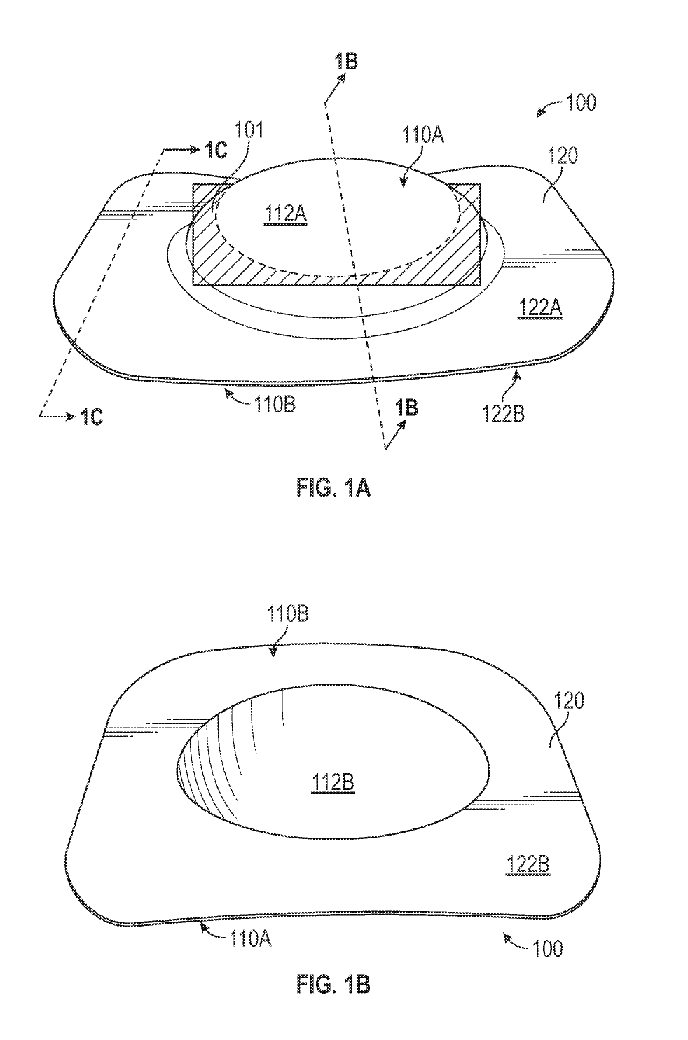

Referring now to the drawings wherein the showings are for purposes of illustrating embodiments of the invention only and not for purposes of limiting the same, FIG. 1A is an elevation view of a first type of pressure applicator 100. The pressure applicator 100 includes a generally round convex portion 112A. The term “generally round”, as used herein, refers to the two-dimensional shape of a cross-section of the convex portion 112A or concave portion 112B and may include circular, elliptical, oval, or egg-shaped patterns for instance. Furthermore, it is contemplated that the cross-section would be taken in an orientation generally parallel to the base of the pressure applicator 100 which, as illustrated by plane 101 of FIG. 1A, coincides with the brim 120 of the applicator 100. FIG. 1A illustrates the generally round convex portion 112A disposed on an inward-facing surface 110A. As used herein the term “inward-facing surface” means a surface that faces toward the wearer when an embodiment is in use. An outward-facing surface 110B is out of view in FIG. 1A but its relative position is indicated by the lead line and arrow extending from reference numeral 110B.

With continuing reference to FIG. 1A, the pressure applicator 100 also includes a brim 120 that forms a generally flat base portion. The brim 120 includes an inward-facing portion 122A, as well as an outward-facing portion 122B. The outward-facing portion 122B is out of view in FIG. 1A but its relative position is illustrated by the lead line and arrow extending from reference numeral 122B.

As shown in FIG. 1A the generally flat base portion may be slightly curved. Such curvature may tend to track the curvature of a wearer's abdomen and thus promote comfort by tending to apply a more even pressure. Such a curvature may also tend to diminish the appearance of a relief pattern in overlaying garments, thereby tending to mask the presence of the device.

FIG. 1B is a perspective view of the pressure applicator 100 as viewed along line 1B-1B. In this view the outward-facing surface 110B and the outward-facing portion 122B of the brim 120 are both visible. FIG. 1B also illustrates a concave portion 112B of the pressure applicator 100. While a concave portion 112B is not required, it may be beneficial in some embodiments because having a convex portion 112A without a concave portion 112B would require a solid structure and thus would require more material. Another embodiment, not shown here, may include a convex portion 112A and concave portion 112B, but the concave portion may be enclosed by extending the brim 120 and the inwardly-facing surface 112B over the concave portion thereby forming a hollow structure. Furthermore, in such hollow embodiments the hollow space may be inflatable to an adjustable degree of firmness. An inflatable structure may be advantageous in some embodiments to further promote comfort.

FIG. 1C is a front view of the inward facing surface 110A of the pressure applicator 100 along line 1C-1C. The convex portion 112A and inward-facing portion 122A of the brim 120 are visible. Also visible are rounded corners 124a, 124b, 124c, and 124d of the brim 120. These rounded corners 124a-d are also visible in FIGS. 1A and 1B but are not labeled. The function of rounded brim corners 124a-d is to improve comfort for the wearer by eliminating pressure points that may tend to dig into the skin, and also to promote masking the presence of the device under clothing. For instance, a pressure applicator having sharp corners may tend to form a relief pattern in an overlaying garment thereby alerting an observer to its presence. Rounded corners 124a-d may tend to diminish such a relief pattern.

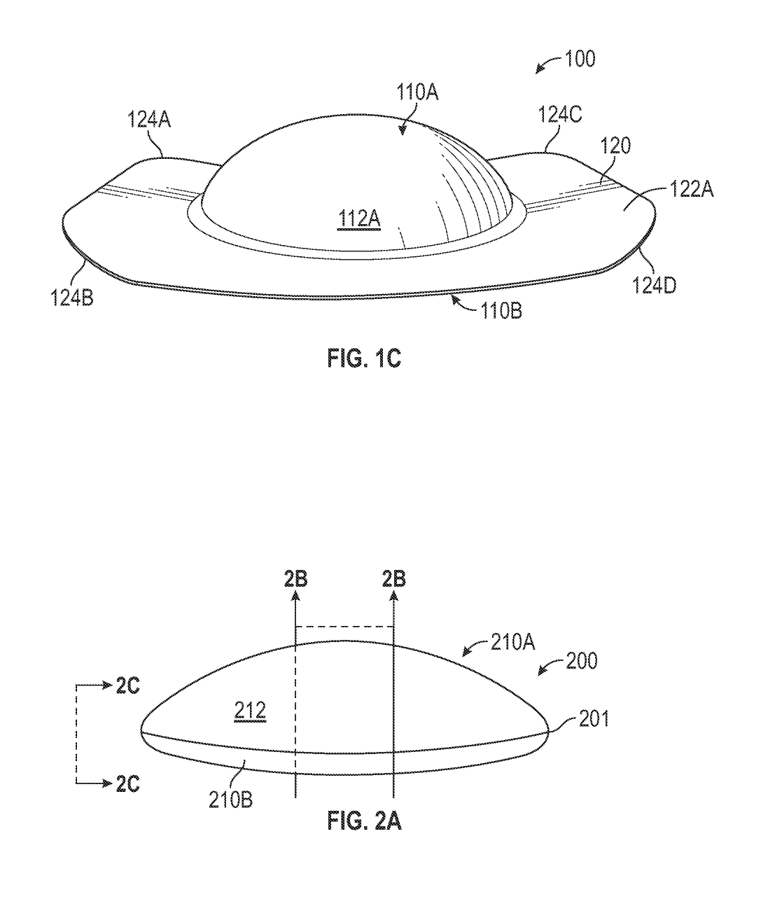

FIG. 2A is an elevation view of another type of pressure applicator 200. One of the more prominent features distinguishing pressure applicator 200 from pressure applicator 100 is that pressure applicator 200 lacks a brim 120. The pressure applicator of FIG. 2A includes an inward-facing surface 210A including a convex portion 212, and an outward-facing surface 210B. The outward-facing surface 210B, which is only partially visible in FIG. 2A, may form a generally flat base similar to that of pressure applicator 100. Similar to pressure applicator 100 the outward-facing surface 210B (i.e. base) may be flat or it may be gently curved so as to approximate the curvature of the wearer's body similar. Furthermore, the inward and outward facing surfaces 210A and 210B may join at a rounded edge 201. The edge may be rounded to a radius sufficient to promote applying a more even pressure to the wearer's body thereby promoting comfort and mitigating against creating excess pressure points that may tend to dig into the flesh or cause irritation.

FIG. 2B is a perspective view of the pressure applicator 200 viewed along line 2B-2B. The outward-facing surface 210B is visible in FIG. 2B. In the illustrated embodiment no concave portion is visible. Accordingly, FIG. 2B illustrates both a solid structure having no concave portion, and a hollow structure having an enclosed concave portion. Similar to the hollow embodiment described previously regarding pressure applicator 100, a hollow embodiment of pressure applicator 200 may be inflatable to an adjustable degree of firmness, which may promote comfort.

FIG. 2C is a front view of the pressure applicator 200 along line 2C-2C. In this view the curvature of an inward-facing surface 210A is visible including a convex portion 212. An outward-facing surface 210B is also partially visible. This perspective illustrates several curvatures of the pressure applicator simultaneously which are indicated by lines C1-C4. Curvature C1 runs laterally from one side to the other, and curvature C4 runs generally fore to aft. Curvatures C1 and C4 pass through apex 203, and are related to each other by a continuous function. Curvature C2 is significantly greater than that of C1 and C4, and comprises a transition from the inward-facing surface 210A to the outward-facing surface 210B.

With continuing reference to FIG. 2C, curvature C3 runs fore-to-aft parallel to C4, but the C3 curvature is less than that of C4. The reason for C4>C3 is that C4 must function to apply pressure to the abdomen of a wearer, which tends to require more curvature. In contrast, the C3 curvature is intended to roughly track the curvature of the wearer's abdomen so as to mask its presence and to promote the application of an even pressure; this requires less curvature. In the embodiment of FIG. 2C, line 205 is directly below apex 203 and represents the lowest point of curvature C3 as well as curvature lines running parallel to C3 on the outward-facing surface 210B. As drawn, the outward-facing surface 210B is curved in two dimensions in the plane in which curvature line C3 lies. However, it is also contemplated that the outward-facing surface 210B may also be curved in three dimensions (not shown) by adding a second curvature (e.g. C5) perpendicular to C3 but lying in the same plane as C3, i.e. similar to the double curvature of the inward-facing surface 210A (i.e. C1+C4).

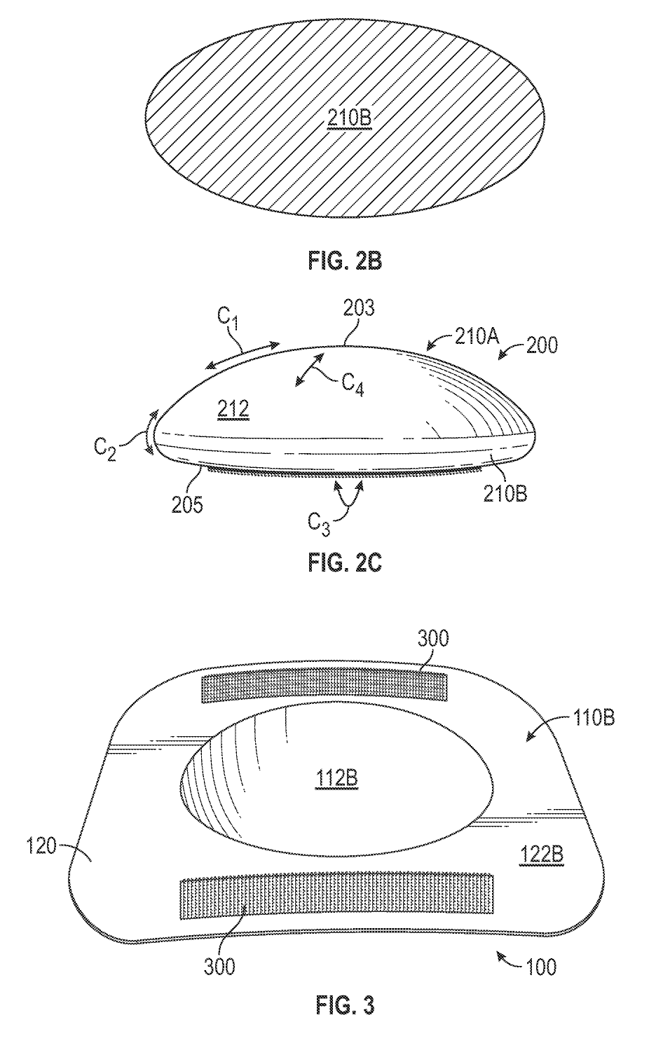

FIG. 3 illustrates the pressure applicator 100 of FIGS. 1A-C equipped with a pair of hook-and-loop fastener strips 300. In the illustrated embodiment, the strips 300 are the hook portion of the hook-and-loop fastener, and they are affixed to the outward-facing surface 110B on the outward-facing portion 122B of the brim 120. The strips 300 are adapted to cooperate with loops inherent in the fabric of a corset which also comprises part of the embodiment. As used herein the term “corset” includes any garment which may be worn about the midsection of a human subject, and which is co-operable with the pressure applicator to produce the desired hunger-suppression effect. Accordingly, it is contemplated that suitable corsets are capable of applying pressure to the midsection. Furthermore, while not shown in the drawings, upon reading the description of the invention herein, it will be understood that the invention is not limited to corset garments. For instance, a pressure applicator 100 or 200, may be adapted to cooperate with a belt to apply pressure in a similar fashion as compared to a corset. What is more important than a garment's classification as a corset or belt is that it is adapted to cooperate with a pressure applicator 100 or 200 to apply pressure to a wearer's abdomen, and allow the pressure applicator to be 100 or 200 to be both reversibly attachable and repositionable relative to the garment.

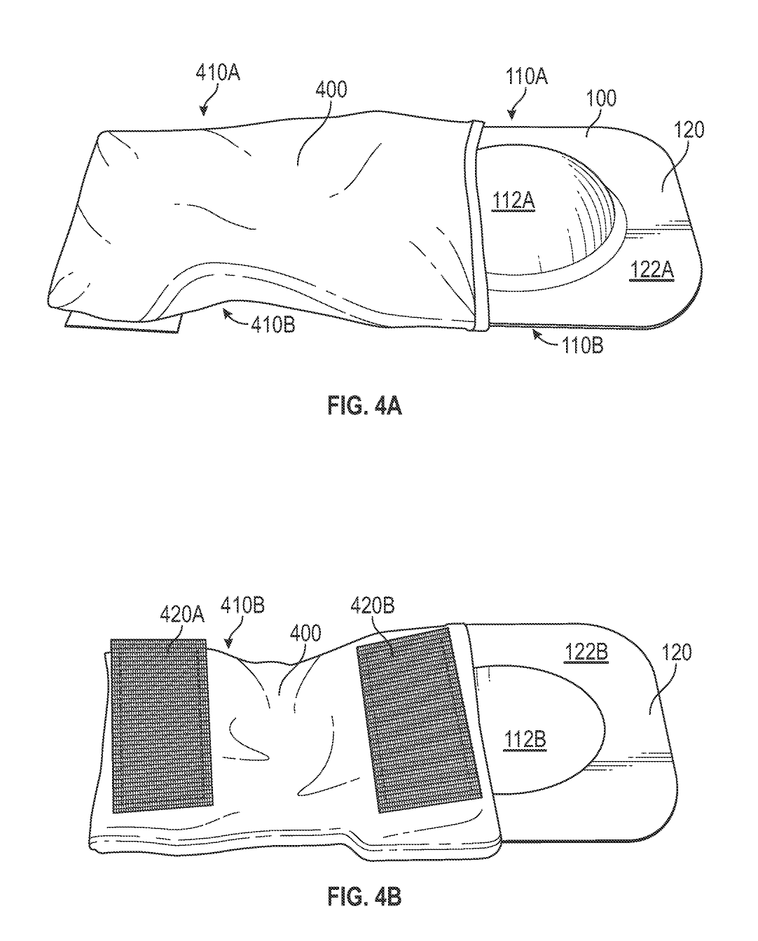

FIG. 4A illustrates an embodiment where the pressure applicator 100 fits into a bag member 400. Similar to the pressure applicator, the bag member 400 has an inward-facing surface 400A and an outward-facing surface 400B. Accordingly, the bag member 400 and pressure applicator 100 have a preferred orientation relative to each other. FIG. 4B illustrates the outward-facing surface 410B of the bag member 400. In the illustrated embodiment, the bag member 400 has a pair of hook fastener strips 420A and 420B affixed to the outward-facing surface 410B, while the pressure applicator 100 has none. Accordingly, in the illustrated embodiment, the pressure applicator 100 may be affixed to a garment by placing it in the bag member 400 in the preferred orientation, and affixing the bag member to the garment.

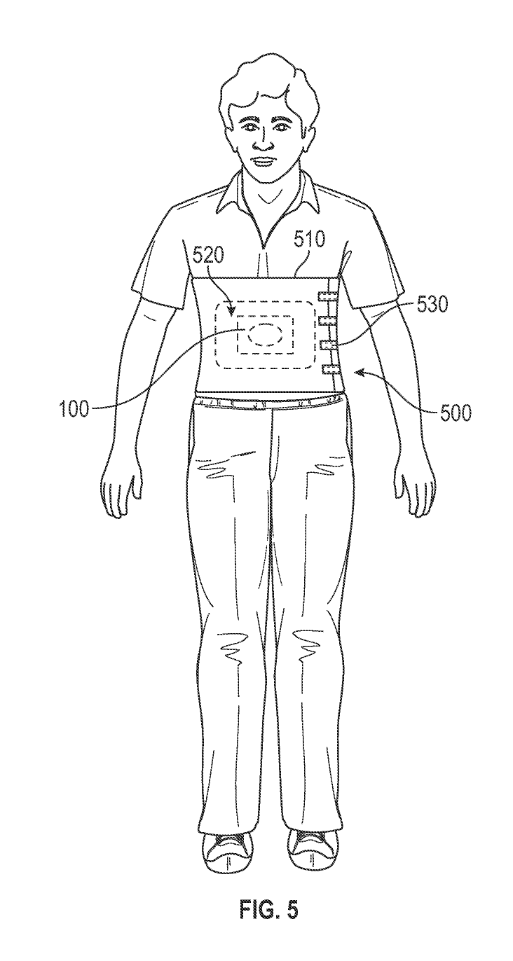

FIG. 5 is an illustration of an embodiment 500, including a pressure applicator 100 and a garment 510. The pressure applicator 100 may be reversibly attached to any of a plurality of positions on the garment 510 according to the wearer's selection. Ordinarily the wearer would position the pressure applicator 100 so as to optimize its hunger-suppression effect and comfort. It is contemplated that the optimal position may vary from one individual to another based on body measurements and variation in internal anatomy. Furthermore, an optimal position for a single individual may vary over time as the individual loses weight. Accordingly, FIG. 5 illustrates a region 520 where a user is likely to desire to attach the pressure applicator 100. This region 520 may therefore be specially equipped with fabric having sufficiently loose fibers to cooperate with and engage the hook fasteners of the pressure applicator 100.

In general, a wide variety of garments may be appropriate provided that they are capable of cooperating with the reversible attachment means provided with a given pressure applicator. For example, a pressure applicator provided with a hook type fastener strip would preferably comprise fabric having fiber loops sufficiently lose to engage the hook fastener. Other suitable means may include snaps, buttons, zippers, ties, clasps, cinches, straps, or any of a wide variety of structures known in the art capable of fulfilling this purpose. Further, the garment should be equipped with means for tightening and loosening the garment such as may be found in a corset. For example, one or more belts or straps may be provided with the garment for this purpose; however, the invention is not limited to belts and straps, but rather is intended to encompass any structure capable of performing the function of tightening and/or loosening the garment including, without limitation, ties, laces, buckles, snaps, and the like. FIG. 5 illustrates a plurality of strap fasteners 530 suitable for adjusting the tightness of the garment 510 abound the waist of a wearer.

It will be apparent to those skilled in the art that the above methods and apparatuses may be changed or modified without departing from the general scope of the invention. The invention is intended to include all such modifications and alterations insofar as they come within the scope of the appended claims or the equivalents thereof.

Having thus described the invention, it is now claimed:

I/We claim:

1. A hunger control device, comprising:
a garment adapted to be worn about a midsection of a human subject; and
a pressure applicator element having a generally flat base portion and a generally round convex portion extending from a plane of the base portion, wherein the pressure applicator is reversibly attachable to a plurality of positions on the garment, and wherein the pressure applicator is positionable between the midsection of the human subject and the garment so that the garment and pressure applicator cooperate to apply pressure to the midsection.

2. The device of claim 1, wherein the pressure applicator further comprises a hook strip of a hook-and-loop fastener structure for reversibly attaching it to the plurality of positions on the garment.

3. The device of claim 2, wherein the garment includes one or more surfaces adapted to engage the hook strip of the hook-and-loop fastener structure in a fastened relation.

4. The device of claim 1, wherein the pressure applicator is receivable by a bag member having a hook strip of a hook-and-loop fastener structure disposed on a surface of the bag member for reversibly attaching it to the plurality of positions on the garment.

5. The device of claim 4, wherein the garment includes one or more surfaces adapted to engage the hook strip of the hook-and-loop fastener structure in a fastened relation.

6. The device of claim 1, wherein the pressure applicator is adapted to minimize or eliminate relief patterns in overlaying garments resulting from its presence under the garment.

7. The device of claim 6, wherein the adaptation to minimize or eliminate relief patterns comprises the base portion of the pressure applicator having a curvature approximating the curvature of a wearer's midsection.

8. The device of claim 7 further comprising rounded corners of the base portion.

9. The device of claim 7. Wherein the pressure applicator lacks a brim structure.

10. The device of claim 1, wherein the garment comprises a corset or a belt.

11. The device of claim 10, wherein the garment includes means for tightening and loosening the garment.

12. The device of claim 11, wherein the garment includes at least one strap for tightening and loosening the garment.

13. A hunger control device, comprising:
a pressure applicator element co-operable with an arbitrary corset or belt, the pressure applicator having a generally flat base portion and a generally round convex portion extending from the plane of the base portion, wherein the pressure applicator is reversibly attachable to a plurality of positions on the garment, and wherein the pressure applicator is positionable between the midsection of the human subject and the garment so that the garment and pressure applicator cooperate to apply pressure to the midsection.

14. The device of claim 13, wherein the pressure applicator further comprises a hook strip of a hook-and-loop fastener structure for reversibly attaching it to the plurality of positions on the arbitrary corset or belt.

15. The device of claim 14, wherein the corset or belt includes one or more surfaces adapted to engage the hook strip of the hook-and-loop fastener structure in a fastened relation.

16. The device of claim 13, wherein the pressure applicator is receivable by a bag member having a hook strip of a hook-and-loop fastener structure disposed on a surface of the bag member for reversibly attaching it to the plurality of positions on the corset or belt.

17. The device of claim 16, wherein the corset includes one or more surfaces adapted to engage the hook strip of the hook-and-loop fastener structure in a fastened relation.

18. The device of claim 13, wherein the pressure applicator is adapted to minimize or eliminate relief patterns in overlaying garments resulting from its presence under the garment.

19. The device of claim 18, wherein the adaptation to minimize or eliminate relief patterns comprises the base portion of the pressure applicator having a curvature approximating the curvature of a wearer's midsection, the base portion having rounded corners, and the base portion lacking a brim structure.

20. A pressure applicator, comprising:
a generally flat base portion;
a generally round convex portion extending from a plane of the base portion;
means for reversibly attaching the pressure applicator to any of a plurality of positions on an arbitrary corset or belt, wherein the pressure applicator is positionable between an abdomen of a human subject and the corset or belt so that the corset or belt and pressure applicator cooperate to apply pressure to the midsection; and
a bag member adapted to receive the generally flat base portion and generally round convex portion, the bag member having a hook strip of a hook-and-loop fastener structure disposed on a surface of the bag member for reversibly attaching it to the plurality of positions on the arbitrary corset or belt;
wherein the pressure applicator is adapted to minimize or eliminate relief patterns in overlaying garments resulting from its presence under the overlaying garment, said adaptation comprising the base portion of the pressure applicator having a curvature approximating the curvature of a wearer's midsection, the base portion having rounded corners, and the base portion lacking a brim structure.
